(19) United States
(12) Patent Application Publication
(10) Pub. No.: US 2025/0268969 A1
(43) Pub. Date: Aug. 28, 2025

(54) OPHTHALMIC COMPOSITION

(71) Applicant: IROMED GROUP S.R.L., Roma (RM) (IT)

(72) Inventor: Ciro CARUSO, Napoli (NA) (IT)

(73) Assignee: IROMED GROUP S.R.L., Roma (RM) (IT)

(21) Appl. No.: 18/251,611

(22) PCT Filed: Oct. 5, 2021
(86) PCT No.: PCT/IB2021/059110

§ 371 Date: May 3, 2023

(30) Foreign Application Priority Data
Nov. 4, 2020 (IT) 102020000026236

(51) Int. Cl. A61K 36/53; A61K 9/00; A61K 9/08; A61K 9/70; A61K 31/05; A61K 31/355; A61K 31/675; A61K 47/32; A61P 31/04; A61P 31/10

(52) U.S. Cl. CPC A61K 36/53; A61K 9/0048; A61K 9/0053; A61K 9/08; A61K 9/7007; A61K 31/05; A61K 31/355; A61K 31/675; A61K 47/32; A61P 31/04; A61P 31/10

(57) ABSTRACT

The present invention refers to combinations, compositions and kits of parts having thymol, or an extract of plant belonging to the genus Thymus containing thymol, and a tocopherol as well as to their use in the prevention and/or treatment of ocular diseases or also for non-therapeutical purposes.

### FIELD OF THE INVENTION

The present invention refers to thymol or extract of plant belonging to the genus Thymus as well as combinations, compositions, formulations and kit comprising thymol or at least an extract of plant belonging to the genus Thymus and also to their use in the prevention and/or treatment of ocular diseases and/or disorders.

### Background

Thyme (for example, Thymus vulgaris L., Lamiaceae; Hossain, AL-Raqmi, AL-Mijizy, Weli and Al-Riyami, 2013), a shrub native to the Mediterranean areas, is a medicinal plant traditionally used in the kitchen as a culinary herb and in pharmacology in the treatment of various diseases such as, for example, affections of the respiratory apparatus (colds, acute bronchitis, asthma, etc.), gastrointestinal disorders (e.g. ulcers), pathologies of the cardiovascular system (e.g. hypertension), pathologies affecting the nervous system (anxiety disorders, depression) by virtue of its sedative properties, but also as an antiseptic, anthelmintic and antifungal agent (see, for example, Al-Bayati, FA (2008). Synergistic antibacterial activity between essential oils of Thymus vulgaris and Pimpinella anisum and methanol extracts. Journal of Ethnopharmacology, 116 (3), 403-406; Giordani, R., Hadef, Y., & Kaloustian, J. (2008). Antifungal compositions and activities of essential oils of some Algerian aromatic plants. Phytotherapy, 79, 199-203; and Fani, M., & Kohanteb, J. (2017). Antimicrobial in vitro activity of Thym essential oil us vulgaris against the main oral pathogens. Journal of Evidence-Based Complementary and Alternative Medicine, 22 (4), 660-666.).

It is also known from the literature that thyme, in particular its main active ingredient, i.e. thymol, has antimicrobial, antiparasitic, antioxidant, anticarcinogenesis, anti-inflammatory and antispasmodic activities, as well as a potential as a growth enhancer and immunomodulator and can increase concentration ability and memory. Other studies have mentioned the use of thyme in the cosmetic and/or therapeutic fields in the treatment or prevention of skin conditions such as wounds, scars, oily skin, acne, hair loss etc., menstrual pain and menopausal problems, water retention, nausea and fatigue. Studies also report that thyme essential oils are among the main used in the food and cosmetics industries as antioxidants and preservatives (Sharifi-Rad, Salehi, Schnitzler, et al., 2017; Zarzuelo & Crespo, 2002).

Thyme contains high concentrations of monoterpenic phenols, including thymol (2-isopropyl-5-methylphenol or 5-methyl-2-(1-isopropyl) phenol or iso-propyl-meta-cresol; C10H14O; 50-64%), carvacrol (iso-propyl-ortho-cresol; 0.4-20.6%), p-cymene (9.1-22.2%), the structures of which are given below, and other monoterpenes such as 1,8-cineol (0.2-14.2%), linalol (2.2-4.8%), borneol (0.6-7.5%), α-pinene (0.9-6.6%) and camphor (0-7.3%) (see, for example, Amiri, H. (2012) Essential oils composition and antioxidant properties of three Thymus species. Evidence-based Complementary and Alternative Medicine, 2012 (728065), 1-8.; Burt, 2004; Nickavar, Mojab and Dolat-Abadi, 2005). Thymol, the main active ingredient of thyme, is a natural monoterpenic derivative of cymene and carvacrol is its isomer.

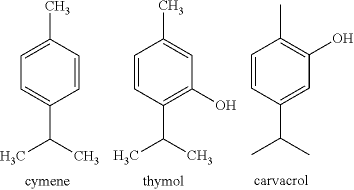

As per what has been said above, it is evident that the use of thyme extract or thymol in therapy is particularly advantageous both because they have numerous therapeutic and beneficial properties and because they are characterized, like most of the active ingredients of plant origin, by an optimal safety profile, i.e. they are free of severe side effects.

However, like many other extracts and active ingredients of plant origin, especially phenolic ones, also thyme extract and thymol are hydrophilic, therefore, despite their pronounced properties found in vitro, in vivo they show a lower or even insignificant activity. Because of their poor liposolubility, in fact, they are unable to cross biological membranes, especially the intestinal and corneal membranes, and this results in poor absorption and therefore poor bioavailability of the active ingredients, especially following oral or topical administration.

Furthermore, since the phytocomplex (i.e. the set of chemical components of a plant, resulting from the natural combination of the active principle with other therapeutically inactive substances (e.g. cellulose) or with different activity, but which globally give the plant the specific therapeutic properties for which it is used) seems to be important for the bioavailability of the main active ingredients, the problem of poor absorption is exacerbated when thymol is administered by itself.

In the literature it is known that bioavailability can be increased with the use of systems of delivery (drug delivery systems) such as liposomes, microspheres, nanoparticles, transferosomes, ethosomes, etc., which can increase the release rate as well as the ability to cross biomembranes rich in lipids. However, these systems have various disadvantages, for example, they are often not very stable and subject to degradation, and require complex and expensive preparation procedures.

Therefore, since the effectiveness of any plant product (or medicament) depends on the ability to deliver an effective dose of active ingredient and given that the drug delivery systems currently available are often expensive and/or difficult to implement, the need is still felt to develop alternative solutions to facilitate the conveyance, especially at the corneal level, of active principles of plant origin and/or plant extracts, such as thymol or thyme extracts, which overcome the drawbacks of currently available systems.

### SUMMARY OF THE INVENTION

Studies carried out by the inventors on an experimental model in vitro and in vivo have surprisingly shown that the topical administration of thymol or thyme extract on the ocular surface can also be useful in the prevention and/or treatment of ocular diseases such as glaucoma, cataracts, keratoconus, ocular dryness or dry eye, conjunctival allergy, conjunctivitis, blepharitis, keratitis, actinic keratitis, stye, UV exposure damage, maculopathy, uveitis, meibomitis, trabeculitis, corneal ulcers, such as infective or traumatic corneal ulcers, optic neuritis etc.

Therefore, the present invention refers to thymol and/or thyme extract for use in the prevention and/or treatment of ocular diseases, in particular those mentioned above.

However, as anticipated above, the main limitation of thyme extract or thymol for topical use is its poor ability to overcome the cornea, which is substantially impermeable to most of the active ingredients due to the presence of gap-junctions that connect cells of the corneal, conjunctiva, sclera and trabecular epithelium, and thus to reach the site of interest in clinically relevant doses.

After extensive experimentation, the inventors have found that the combination of at least one extract of plant belonging to the genus Thymus or thymol, with tocopherol or derivatives thereof, for example salts and/or esters of tocopherol, can increase the ocular bioavailability of thymol or thyme extract beyond the cornea.

Said combination would therefore guarantee the passage of the active principle thymol into the posterior segment of the eye (vitreous chamber) which thus can also reach the retina and the optic nerve, providing a valid solution to the clinical problem of ocular diseases and, in particular, of ocular infections.

Therefore, the present invention refers to tocopherol for the use as adjuvant and/or enhancer of corneal bioavailability of thymol in a therapy based on extract of at least a plant belonging to the genus Thymus or thymol. In other words, the invention refers to the use of tocopherol for enhancing the corneal bioavailability of the active ingredient thymol or of an extract of plant belonging to the genus Thymus comprising said active ingredient.

The present invention refers also to the combination of at least an extract of plant belonging to the genus Thymus, or thymol, and tocopherol or derivatives thereof.

The present invention also refers to compositions, preferably for topical use, more preferably in form of collyrium, comprising the combination of at least an extract of plant belonging to the genus Thymus or thymol, tocopherol or derivatives thereof and one or more suitable carriers as well as to kit of parts comprising the aforementioned compounds separated and independently formulated in suitable dosage form for sequential or simultaneous administration of the various compounds.

Moreover, the present invention relates to the use of the aforementioned combination, compositions and kits in the prevention and/or treatment of ocular diseases such as, for example, glaucoma, cataract, keratoconus, ocular dryness or dry eye, conjunctival allergy, conjunctivitis, blepharitis, keratitis, actinic keratitis, stye, damage from UV exposure, maculopathy, uveitis, meibomitis, trabeculitis, corneal ulcers, such as infective or traumatic corneal ulcers and optic neuritis and/or to the non-therapeutic or cosmetic use of the aforementioned combination, compositions and kits, for example, as an ocular lubricant, artificial tears, liquid for contact lenses, liquid for cleaning and/or disinfection of the eyelids, for example in the form of spray or medicated bandages, liquid for cleansing and/or disinfection of the mouth and nostrils.

Thanks also to the synergy of its components, the combination object of the present invention allows to obtain at the same time the reduction of various symptoms associated with ocular diseases, such as a reduction of the intraocular pressure, lens opacity, conjunctival hyperemia, dry eye, the sensation of a foreign body and/or pain, photophobia, red eye, lacrimation, secretion and blurred vision, but above all to reduce the dosage and daily dosage, with a consequent increase in patient compliance. Other advantages and features of the present invention will be apparent from the following detailed description.

### Glossary

The terms used in the present description are as generally understood by a person skilled in the art, except where otherwise indicated.

With the term “extract′, in the context of the present description, is meant any product attributable to a plant drug including all products derived from mechanical treatments (pulverization, grinding, mixing and/or other methods) or from extractive treatments (solvent extraction, distillation, maceration and/or other specific methods) operated on a drug, including mother tinctures and essential oils.

In the context of the present description, the term “effective amount′ means amount of active compound, combination or composition comprising the compounds of the invention, high enough to provide the desired benefits and at the same time low enough not to cause serious side effects.

With the wording “bioavailability enhancer”, “absorption enhancer” or “biopotentiator”, in the context of the present description are intended molecules, substances, compounds or agents which per se may or may not have their own pharmacological activity but, when used in combination with an active principle, they are able to increase the activity of the latter in different ways, for example, by increasing its absorption through biological membranes, by enhancing the active principle by conformational interaction, by acting as a receptor for the active principle or making the target cells more receptive to the active ingredient. A “biopotentiator” is therefore a molecule, substance, compound or agent capable of enhancing the bioavailability and effectiveness of a particular active ingredient with which it is combined, exercising, or even without exercising, a pharmacological activity per se. They can also be functional excipients included in the formulation to increase the absorption of a pharmacologically active molecule. A biopotentiator can therefore be used as an adjuvant a therapy based on a specific active principle which benefits, for example in terms of speed and extent of absorption, bioavailability and/or efficacy, from the presence of said biopotentiator. In the context of the present description, with the terms bioavailability enhancer or biopotentiator particular reference is made to tocopherol or its derivatives.

The terms “tocopherol or derivatives thereof”, “derivatives of tocopherol” or “tocopherol derivatives” in the context of the present description mean derivatives or analogues of vitamin E, in particular pharmaceutically or physiologically acceptable salts and esters of tocopherol as well as substitution products thereof.

With the wording “drug-extract ratio” or with the abbreviation “DER”, in the context of the present description, is meant the relationship between the amount of drug used and that of extract obtained. In other words, DER can also be defined as “extractive force” because it represents the ratio between the weight of the starting dry plant material and the weight (dry extract) or volume (liquid extract) of the final preparation. The DER can also be used for the calculation of the equivalent dose. For example, if the recommended daily dose of a plant substance is equal to “1.5 g of dry plant substance or equivalent” and a 1:3 DER liquid extract of the same substance is available, it will be necessary to administer 4.5 mL of liquid extract (1.5 g×3=4.5).

With the term “comprising”, in the context of the present description, also “consisting essentially of” or “consisting of′ is meant.

In the context of the present description, “about” refers to the experimental error which may occur during conventional measurements. More specifically, when it refers to a value it indicates+5% of the indicated value and when referred to a range+5% of the extreme points thereof.

### DETAILED DESCRIPTION OF THE INVENTION

As mentioned above, the present invention refers to a composition comprising, or consisting of, thymol or at least an extract of plant belonging to the genus Thymus with tocopherol or its derivatives, where with tocopherol derivatives also pharmaceutically and/or physiologically acceptable salts and esters of tocopherol are meant.

With the wording “at least an extract of plant belonging to the genus Thymus” in the context of the present description it is meant that the composition can comprise at least one or more extracts of plant belonging to the genus Thymus, i.e. at least one or more thyme extracts. The extract or extracts can be selected, for example, among Thymus vulgaris L. extract, Thymus zygis extract, Thymus striatus extract, Thymus spinulosus extract, Thymus richardii extract, Thymus catharinae extract, Thymus herba-barona extract, Thymus odoratissimus extract, Thymus oenipontanus extract, Thymus kosteletzkyanus extract, Thymus alpestris extract, Thymus pulegioides extract, Thymus longicaulis extract, Thymus praecox extract, Thymus thracicus extract and mixtures thereof, and it is preferably Thymus vulgaris L. extract.

The extract may be an extract obtained by processing one or more parts of the plant, for example aerial parts of the plant, according to any of the procedures known in the state of the art. Suitable extracts for the purposes of this description may be liquid (aqueous, alcoholic or hydroalcoholic) or dry extracts, mother tinctures, essential oils or mixtures thereof such as, for example, those mentioned in the monograph of the European Medicines Agency (EMA) entitled Community herbal monograph on Thymus vulgaris 7 and Thymus zygis L., herba (https://www.ema.europa.eu/en/documen s/erbal-monograph/final-community-herbal-monograph-herba en.pdf), just to name a few:
liquid extract such as, for example, a. liquid extract (DER 1:1) in ethanol, preferably 24% (V/V); b. liquid extract (DER 1:1) in mixture of glycerol and ethanol, preferably glycerol 85% (m/m) and ethanol 25% (m/m), 0.1:2; c. liquid extract (DER 1:2-2.5) in mixture of ammonium solution, glycerol, ethanol and water, preferably ammonium solution 10% (m/m): glycerol 85% (m/m): ethanol 90% (V/V): water (1:20:70:109) or d. liquid extract (DER 1:4.5) in mixture of ammonium solution 10% (m/m): glycerol 85% (m/m): ethanol 96% (V/V): water (1.2:25:112:113); e. liquid extract of fresh aerial parts of the plant (DER 1:1.5-2.4) in water;
tinctures such as, for example, f. tincture (1:10 or 1:5) in ethanol preferably at 70% (V/V);
dry extracts such as, for example, g. dry extract (DER 6-10:1) in ethanol preferably 70% (v/v); h. dry extract (DER 1,6-2,4:1) in ethanol preferably 96% (V/V); i. dry extract (DER 7-13:1) in water; I. dry extract (DER 1:4) in water and
essential oils.

According to an embodiment, the extract can be an essential oil of thyme prepared by extraction of aerial parts of the plant, in particular leaves, dried or fresh in an ethanol/water mixture, preferably at a temperature of 22° C. According to an embodiment, the extract can be a dry extract of thyme, in particular of Thymus Vulgaris L., obtained by aqueous extraction (DER 1:4) of aerial parts of the plant, in particular dry or fresh tops, and subsequent drying.

Therefore, the present invention also refers to a combination comprising extract of at least one plant belonging to the genus Thymus, or thymol, and tocopherol, in which said extract of at least one plant belonging to the genus Thymus can be obtained by extracting aerial parts of the plant, in particular leaves, dry or fresh in an ethanol/water mixture, preferably at a temperature of 22° C. or also by aqueous extraction (preferably DER 1:4) of aerial parts of the plant, in particular tops, dry or fresh, and subsequent drying. As will be evident to a person with general knowledge in the field, other extraction procedures known in the art are however suitable.

Moreover, even if for the purposes of the present invention, extracts of thyme prepared according to the methods described above are particularly suitable, also extracts of thyme commonly available on the market are however suitable, for example, the essential oil of thyme marketed by ACEF, the liquid extract marketed under the name of Thyme herb (tops) fluid extract) (22° (nVh Italy) or the dry extract marketed under the name of Thyme tops dry extract D/E 4:1 (nVH Italy).

Thyme extract, in particular thyme essential oil, will preferably be titrated in thymol (i.e. it comprises a concentration of 5-methyl-2-(1-isopropyl) phenol) between 30 and 60% w/w, preferably between 35 and 45% w/w, more preferably 40-42.5% w/w; and/or titrated in, i.e. comprising a concentration of, p-cymene between 30 and 60% w/w, preferably between 35 and 45% w/w, more preferably at 40-42.5% w/w; and/or titrated in alpha-pinene between 5 and 10% w/w, preferably between 6 and 7% w/w; and/or titrated in camphene between 1 and 5% w/w, preferably between 2 and 3% w/w or titrated in terpinolene between 1 and 5% w/w, preferably between 2 and 3% w/w; and/or titrated in linalool between 1 and 5% w/w, preferably between 2 and 2.5% w/w; and/or titrated in beta-pinene between 0 and 2% w/w, preferably between 0.5 and 0.6% w/w.

It is also understood that if, for the preparation of the thyme extract, not one but more species (for example two, three, four, five, six, seven, eight or more species) of plant belonging to the genus Thymus are used in mixture it will be sufficient that the titers, in particular the thymol titer, are satisfied for the total mixture.

Therefore, the present invention also refers to a combination comprising extract of at least one plant belonging to the genus Thymus, or thymol, and tocopherol, in which said extract of at least one plant belonging to the genus Thymus is titrated in thymol between 30 and 60% w/w, preferably between 35 and 45% w/w, more preferably 40-42.5% w/w. In the embodiments that provide for the use of thymol, thymol can be obtained by means of synthesis processes known in the art, for example by reaction in the gas phase between meta cresol and propene:

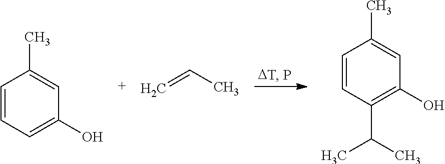

or by extraction from a plant belonging to the genus Thymus, in particular Thymus vulgaris L., according to any one of the conventional methods known in the art, for example one of the extraction methods described above and subsequent purification.

Thyme extract, in particular of Thymus vulgaris L., can be administered for example in an amount comprised between 0.00005 μg/mL and 600 μg/mL, in particular between 0.0005 and 500 μg/mL, preferably in 1-2 separate administrations (for example 0.1 μg×2 times per day, or 0.2 μg 1 time a day).

With “tocopherol′ in the context of the present description is meant tocopherol or a derivative thereof for example pharmaceutically and/or physiologically acceptable salt or ester thereof.

Tocopherol derivatives particularly useful for the purposes of the present invention are tocopherol esters with a carboxylic acid such as, for example, tocopherol succinate, tocopherol acetate, tocopherol linoleate and tocopherol phosphate, but also polyoxyalkylene glycol esters of tocopherol esters such as, for example, alpha tocopheryl polyoxyethylene glycol (1000) succinate (hereinafter also VE-TPGS). By way of example, but in no way limiting, tocopherol derivatives useful for the purposes of the present invention can be selected from: polyoxyethylene glycol esters of tocopherol esters with a carboxylic acid, preferably wherein said tocopherol ester with a carboxylic acid is selected from tocopherol succinate, tocopherol acetate, tocopherol linoleate and tocopherol phosphate and/or preferably wherein the polyoxyethylene glycol function has a molecular weight between about 600 Da and about 6000 Da, preferably between about 600 Da and about 1500 Da. According to one embodiment, the preferred ester is alpha tocopheryl polyoxyethylene glycol (1000) succinate, i.e. a polyoxyethylene glycol ester of alpha-tocopheryl succinate in which the polyoxyethylene glycol fraction of the molecule has an average molecular weight of about 1000 Da.

Therefore, the present invention refers to a combination comprising extract of at least one plant belonging to the genus Thymus, as defined above, or thymol, with tocopherol, in which said tocopherol is an ester of alpha-tocopherol, preferably selected from tocopherol succinate, tocopherol acetate, tocopherol linoleate and tocopherol phosphate or mixtures thereof. The present invention also refers to a combination comprising extract of at least one plant belonging to the genus Thymus, as defined above, or thymol, with tocopherol, wherein said tocopherol is a polyoxyalkylene glycol ester of an ester of alpha-tocopherol with a carboxylic acid, preferably wherein said alpha-tocopherol ester with a carboxylic acid is selected from tocopherol succinate, tocopherol acetate, linoleate tocopherol and tocopherol phosphate or mixtures thereof. The present invention also refers to a combination comprising extract of at least one plant belonging to the genus Thymus, as defined above, or thymol, with tocopherol, wherein said tocopherol is a polyoxyalkylene glycol ester of an ester of alpha-tocopherol with a carboxylic acid, preferably wherein the polyoxyethylene glycol function has a molecular weight between about 600 Da and about 6000 Da, preferably between about 600 Da and about 1500 Da, and is preferably d-alpha-tocopherol-polyethylene glycol 1000 succinate (VE-TPGS).

VE-TPGS can be prepared according to any of the methods known in the art, for example, by esterification of alpha-tocopherol succinate with polyethylene glycol 1000 or according to any of the methods described in U.S. Pat. No. 2,680,749 (Cawley et al.) Or also readily available on the market, for example, VE-TPGS from Isochem (Isodel®).

Tocopherol, in particular VE-TPGS, can be administered for example in amounts ranging from 0.00001 μg/mL to 600 μg/mL, in particular between 0.0001 and 500 μg/mL, preferably in 1-2 separate administrations (for example 0.1 μg×2 times a day, or 0.2 μg 1 time a day).

In particular, the inventors have shown that it is possible to successfully use amounts equal to about 0.001-0.400 μg per day of tocopherol, preferably VE-TPGS, in combination with thymol or with an extract of at least one plant belonging to the Thymus genus which comprises thymol as described here, obtaining, among other beneficial effects, also a significant increase in the bioavailability of thymol in the treated subjects.

The skilled in the art will be able to adapt the amount of the extracts used and/or the compounds used in the preparation of the combinations and/or compositions to be administered according to the needs. The attending physician will also be able to identify the optimal dosage for the subject to be treated based on age, sex, weight, general state of health and also based on the pathology to be treated. Therefore, the dosage of the single components can also be adapted during the period of taking the combination or composition of the invention depending on the results obtained over time.

Although it is not an essential feature of the invention, the inventors have identified the optimal weight ratio between the components of the combination. Preferably, the weight ratio between extract of at least one plant belonging to the genus Thymus, or thymol, and tocopherol is between 0.001:4 and 0.01:3, and is preferably 0.1. Optional active ingredients that may be included in the combination of the present invention according to any of the embodiments described herein include, but are not limited to, the racemic and enantiomeric forms, the pharmaceutically and/or physiologically acceptable salts and esters of the following compounds: beta-blockers (for example betaxolol, timolol, and carteolol); alpha-agonists (for example, apraclonidine); carbonic anhydrase inhibitors; dopaminergic agonists and antagonists; cholinergic drugs (for example, pilocarpine and carbachol); prostaglandins and prostaglandin derivatives; ACE inhibitors; steroids (for example glucocorditicoids); calcium channel blockers; antihypertensives; non-steroidal anti-inflammatory drugs (for example diclofenac, flurbiprofen, ketorolac, indomethacin and ketoprofen); steroidal anti-inflammatories (for example fluorometolone, dexamethasone, prednisolone, tetrahydrocortisol and triamcinolone); antibiotics such as aminoglycosides (for example, tobramycin); quinolones (for example, ciprofloxacin and ofloxacin); beta-lactams (for example, cephalosporins such as cefamandole); antifungals, such as nafamycin; antivirals, such as aciclovir and ganciclovir; anti-cataract agents; antioxidants (for example, Vitamin A, Vitamin C, selenium, carotenoids, lycopene, coenzyme Q-10, lipoic acid and their mixtures); antihistamines; antimetabolites (for example, 5-fluorouracil (5-FU) and methotrexate); immunosuppressants, such as cyclosporine and lenflunimide; growth factors such as EGF, FGF, PDGF; vitamins (for example riboflavin 5 phosphate); prodrugs of the drug classes described above or mixtures thereof.

Therefore, the present invention also refers to a combination comprising, or consisting of, extract of at least one plant belonging to the genus Thymus, preferably Thymus vulgaris L., or thymol, with tocopherol or a derivative thereof, preferably VE-TPGS, and optionally one or more further active ingredients as defined above, or selected from the group consisting of: beta-blockers; alpha-agonists; carbonic anhydrase inhibitors; dopaminergic agonists and antagonists; cholinergic drugs; prostaglandins; ACE inhibitors; steroids; calcium channel blockers; antihypertensives; non-steroidal anti-inflammatory drugs; steroidal anti-inflammatory drugs; antibiotics; antifungals; antivirals; anti-cataract agents; antioxidants; antihistamines; antimetabolites; immunosuppressants; growth factors, vitamins or mixtures thereof. According to a preferred embodiment, the combination comprises, or consists of, an extract of at least one plant belonging to the genus Thymus, preferably Thymus vulgaris L., or thymol, with tocopherol or a derivative thereof, preferably VE-TPGS, and riboflavin 5 phosphate, the latter particularly useful for its protective action against UV rays.

According to an embodiment, the combination comprises at least one extract of plant belonging to the genus Thymus, preferably Thymus vulgaris L. extract, or thymol, tocopherol and one or more active ingredients as defined above as the only active ingredients in the treatment and/or in the prevention of ocular diseases in particular glaucoma, cataract, keratoconus, ocular dryness or dry eye, conjunctival allergy, conjunctivitis, blepharitis, keratitis, actinic keratitis, sty, damage from UV exposure, maculopathy, uveitis, meibomite, trabeculitis, corneal ulcers e.g. corneal ulcers from infection or trauma, optic neuritis, etc. In this embodiment, the combination may possibly also include other active ingredients, provided that said other active ingredients are not indicated in the treatment of ocular diseases.

According to another embodiment, the combination comprises at least one extract of plant belonging to the genus Thymus, preferably Thymus vulgaris L. extract, or thymol, and tocopherol as the only active ingredients in the treatment and/or prevention of ocular diseases. In this embodiment, the combination may possibly also include other active ingredients, provided that said other active ingredients are not indicated in the treatment of ocular diseases.

The present invention also refers to composition comprising the combination according to any one of the embodiments herein described and one or more suitable carriers and/or excipients.

The inventors have also observed that the greater effectiveness of the combination, also by virtue of the greater synergy between the various active ingredients, occurs when in the composition:
the plant extract belonging to the genus Thymus, or thymol, is present in a concentration between 0.001 and 1% w/w, preferably 0.01 and 0.1% w/w, more preferably 0.05% w/w; and
tocopherol or its derivatives are present in a concentration comprised between 0.01 and 3% w/w, preferably 0.1 and 2, more preferably 0.5% w/w.

Therefore, the present invention also refers to a composition wherein the plant extract belonging to the genus Thymus, or thymol, is present in a concentration between 0.001 and 1% w/w, preferably 0.01 and 0.1% w/w, more preferably 0.05% w/w and/or wherein tocopherol or its derivatives are present in a concentration ranging from 0.01 to 3% w/w, preferably 0.1 and 2, more preferably 0.5% w/w.

Suitable carriers for the purposes of the present invention are those commonly known in the art as suitable for topical administration, in particular ocular administration for example, a buffered isotonic solution at pH 6.7-7.2.

According to an embodiment, the composition comprises at least one extract of plant belonging to the genus Thymus, preferably Thymus vulgaris L. extract, or thymol, tocopherol and, optionally, one or more optional active ingredients as defined above, as the only active ingredients in the treatment and/or prevention of ocular diseases. In the latter case, the composition may include other components, such as excipients, carriers, stabilizers, preservatives and the like and/or other active ingredients, provided that said other active ingredients are not indicated in the treatment of ocular diseases.

The compositions of the present invention may comprise other components, for example, excipients, carriers, formulation aids such as: buffers (for example, phosphate, borate and citrate), chelating agents (for example EDTA), preservatives, (for example, benzalkonium chloride) tonicity agents (for example, sodium chloride and mannitol). The compositions of the present invention may also include viscosity modifying agents such as, for example: cellulose esters, (hydroxypropylmethylcellulose (HPMC), hydroxyethylcellulose (HEC), ethylhydroxyethylcellulose, hydroxypropylcellulose, methylcellulose and carboxymethylcellulose; carbohydrates (polyvinyl glycol); carrageenans; and guar, agarose, tragacanth and xanthan gum. The concentration of said viscosity modifying agents can vary between about 0.1 and about 5%, but said formulations will generally have a viscosity between about 10 and about 1000 centipoises.

The addition of polyvinyl alcohol (PVA) is particularly preferred since, without wishing to be bound by any theory, it has been noted that PVA favors the solubilization of the active ingredients and also a faster repair of the corneal epithelium, an effect that is found also with the administration of the combination of thyme extract or thymol with tocopherol or its derivatives according to any of the embodiments described here but which is accentuated and enhanced by the addition of PVA.

Therefore, the present invention also refers to a composition comprising the combination of at least one extract of plant belonging to the genus Thymus or thymol with tocopherol or its derivatives according to any of the embodiments described here and PVA, preferably in the form of eye drops or in a buffered isotonic solution at pH 6.7-7.2.

The compositions according to any of the embodiments provided in the present description can be formulated in any form and by any route of administration and associated with any other component, in a variety of ways.

According to an embodiment, the compositions of the invention are compositions for topical use in liquid, semi-liquid, semi-solid or solid form such as, for example, collyrium, solution, sterile solution, suspension, eye drops, spray, ophthalmic bath, gel, salve, ointment, cream, medicated wipe, occlusive bandage (e.g. in the form of medicated gauze), ophthalmic inserts (e.g. medicated controlled release hydroxypropylcellulose inserts).

Therefore, the present invention also relates to the combination or composition according to any of the embodiments described above in the form of collyrium, solution, sterile solution, suspension, eye drops, spray, ophthalmic bath, gel, salve, ointment, cream, medicated wipe, occlusive bandage and ophthalmic inserts. According to a preferred embodiment, the composition for topical use according to the present invention will be in the form of eye drops or isotonic solution buffered at pH 6.7-7.2.

According to another embodiment, the compositions of the invention are compositions for oral use in solid form such as, for example, capsules, soft capsules, tablets, pills, tablets, jellies, powders or granules or liquid such as emulsions, solutions, prepared or impromptu suspensions, syrups and elixirs. Therefore, the present invention also refers to the combination or composition according to any of the embodiments described above in solid form, preferably in the form of capsules, soft capsules, tablets, pills, tablets, jellies, powders or granules or in liquid form, preferably emulsions, solutions, prepared or extemporaneous suspensions, syrups and elixirs. In the case of solid forms, the excipients will preferably be selected from, but not limited to, the following: a) carriers, such as sodium citrate and calcium phosphate, b) fillers such as starch, lactose, microcrystalline cellulose, sucrose, glucose, mannitol, triglycerides and colloidal silica, c) humectants, such as glycerol, d) disintegrating agents, such as alginates, calcium carbonate, starches, derivatives of starch, cellulose and polyvinylpyrrolidone, silicates and sodium carbonate e) binders such as carboxymethylcellulose, alginates, gelatin, polyvinylpyrrolidone, sucrose, polymeric derivatives of cellulose, starch derivatives f) retardants such as paraffin, cellulose polymers, esters of fatty acids g) accelerators of absorption, such as quaternary ammonium compounds, h) wetting agents and surfactants such as cetyl alcohol and glycerol monostearate, i) adsorbents, such as benthic clays and kaolin, k) lubricants such as talc, calcium stearate, magnesium stearate, polyethylene glycol, sodium lauryl sulfate, sodium stearyl fumarate j) glidants such as talc, colloidal silica.

The solid dosage forms, such as tablets, capsules, soft capsules, gelatins, pills and granules, may optionally be coated with enteric, gastric or other coatings known in the state of the art, possibly allowing the release of the active ingredients only or preferably in a certain part of the intestine, possibly in a delayed way. Substances which may permit such delayed use include, but are not limited to, polymers and waxes.

In the case of liquid forms, suitable excipients include in particular, but are not limited to, diluents such as water or other solvents, solubilizing and emulsifying agents selected from ethyl alcohol, polyalcohols, propylene glycol, glycerol, polyethylene glycol and sorbitan esters. These formulations may also contain sweeteners and flavorings.

According to an embodiment, the compositions according to the present invention do not contain excipients and/or other additives, but only the active ingredients and/or compounds according to any of the embodiments described here, dissolved or suspended in a suitable carrier. However, where necessary, suitable excipients can be selected from those normally known in the state of the art and include, but are not limited to: surfactants (for example sodium lauryl sulfate and polysorbates), adsorbents (for example silica gel, talc, starch, bentonite, kaolin), glidants and anti-adhesives (e.g. talc, colloidal silica, corn starch, silicon dioxide), antioxidants, binders (e.g. gums, starch, gelatin, cellulose derivatives, sucrose, sodium alginate), plasticizers (e.g. ethyl cellulose and other cellulose derivatives, acrylates and methacrylates, glycerol and sorbitol), preservatives (e.g. parabens, sulfur dioxide, polyhexamethylene biguanide or PHMB), edetate disodium (EDTA), viscosifiers, emulsifiers, humectants, wetting agents, chelators, polyvinyl alcohol and mixtures thereof.

By way of example, the composition may comprise one or more extracts of thyme or thymol, tocopherol and, optionally, one or more optional active ingredients as defined above, in a buffered isotonic solution at pH 7.0, preferably physiological solution or isotonic saline based on 0.9% w/v sodium chloride.

The composition, according to any embodiment of the present invention will, for example, be a pharmaceutical, nutraceutical, cosmetic, dietary and/or nutritional composition, a medical device, a food supplement, a food product, a medicament, a medicated food or a food for special medical purposes.

The compositions according to any of the embodiments described here can be used in any subject for which the administration of thymol and/or thyme extract is indicated but in particular in any subject affected by the aforementioned ocular diseases, also in the veterinary field.

The combination of the aforementioned active ingredients can be used formulated in a single composition according to the various embodiments described above or in a kit of parts which comprises the different separate ingredients, for example, in single compositions formulated in suitable dosage form for topical or oral use as defined above, for the sequential or simultaneous administration of the various active ingredients and/or compounds.

Therefore, the present invention also refers to kits of parts which comprise the various active ingredients of the combination according to any of the embodiments described herein separated and formulated in a suitable dosage form for sequential or simultaneous administration of the various active ingredients. The present invention relates, for example, to a kit of parts comprising a first composition for topical or oral use which comprises at least one extract of plant belonging to the genus Thymus, or thymol, a second composition for topical or oral use which comprises tocopherol, preferably alpha-tocopherol, more preferably VE-TPGS, and optionally also a third composition for topical or oral use comprising one or more of the optional active ingredients as defined above, for use in the prevention and/or treatment of ocular diseases, by the sequential or simultaneous administration of said first, second, or optionally third, composition.

As will also be evident from the following examples, the combination of the present invention has proved to be particularly effective in the prevention and treatment of ocular infections, such as for example chalazion, sty, blepharitis, keratitis, actinic keratitis, conjunctivitis, uveitis, endophthalmitis etc. In particular, the combination has proven effective in the prevention and treatment of ocular infections caused and/or sustained by Pseudomonas aeruginosa and Acanthamoeba castellanii.

This represents a further particularly significant advantage of the present invention since the thyme extract or thymol has proven to be more effective, or at least as effective as the synthetic antibiotics and antiseptics known in the art such as, for example, chlorhexidine. Therefore, the present invention also refers to extract of at least one plant belonging to the genus Thymus or thymol, optionally in combination with tocopherol or its derivatives, according to any of the embodiments described herein for use in the treatment of ocular infections, in particular ocular infections caused and/or sustained by Pseudomonas aeruginosa and Acanthamoeba castellanii.

As will be evident to those skilled in the art from the examples provided below, the present invention constitutes a valid support in the reduction or resolution of the symptoms associated with the ocular diseases mentioned above, in particular glaucoma, cataract, keratoconus, ocular dryness or dry eye, allergy conjunctival, conjunctivitis, blepharitis, keratitis, actinic keratitis, stye, damage from UV exposure, maculopathy, uveitis, meibomitis, trabeculitis, corneal ulcers, for example corneal ulcers from infection or traumatic, optic neuritis, more particularly maculopathy, uveitis, dry eye, meibomite, trabeculitis, corneal ulcers, for example corneal ulcers due to infection or traumatic, and optic neuritis, thanks to the combined and diversified action of its components, but above all thanks to the adjuvant action of tocopherol which acts as an enhancer of the bioavailability of thymol and other active ingredients included in thyme extract.

Therefore, the present invention also refers to tocopherol or its derivatives as defined above for use as an adjuvant and/or enhancer of the corneal bioavailability of thymol in a therapy based on the extract of at least one plant belonging to the genus Thymus or thymol. In other words, the invention refers to the use of tocopherol to increase the corneal bioavailability of the active ingredient thymol or of an extract of plant belonging to the genus Thymus comprising said active ingredient.

The action of the individual components and of the combination object of the present invention has been evaluated by means of in vitro and/or in vivo tests which, moreover, have shown how the composition of the invention is particularly effective in the treatment of ocular pathologies thanks to the reinforcement (synergistic) action of its components, but above all thanks to the adjuvant action of tocopherol which acts as an enhancer of the bioavailability of thymol and of the other active ingredients included in the thyme extract.

Therefore, the present invention refers also to the combination, compositions and kit, according to any one of the embodiments described above, for the use as a medicament.

Furthermore, the present invention also refers to the combination, to the compositions and to the kit, according to any of the embodiments described herein, for use in the prevention and/or treatment of ocular diseases, preferably selected from glaucoma, cataract, keratoconus, ocular dryness or dry eye, conjunctival allergy, conjunctivitis, blepharitis, keratitis, actinic keratitis, sty, UV damage, maculopathy, uveitis, meibomitis, trabeculitis, corneal ulcers, for example, infective or traumatic corneal ulcers, and optic neuritis, in both humans and animals.

In addition to this, the object of the present invention is also the non-therapeutic or cosmetic use of the combination, composition, or kit according to any of the embodiments described above, for example, as an ocular lubricant, artificial tears, liquid for contact lenses, liquid for cleaning and/or disinfection of the eyelids, for example in the form of sprays or medicated bandages, liquid for cleaning and/or disinfection of the mouth and nostrils, etc. The combination of the aforementioned active ingredients can be formulated in a single composition or in a kit according to the various embodiments described above and prepared for example by mixing the active ingredients selected in a carrier suitable for topical ocular administration with any other active ingredients and/or excipients as known to those skilled in the art.

### Examples

Some non-limiting examples of the compositions according to the present invention are reported below. Modifications or variations of the embodiments exemplified herein, obvious to one skilled in the art, are encompassed by the appended claims.

### EXAMPLE 1: Collyrium, Solution Based on Extract of Thymus vulgaris L

### Composition A

| Supplier + code | Qualitative composition | g in 100 g |
|---|---|---|
| Alsa Lab | Production Water | q.s. to 100 |
| Acef 002925 | Riboflavin 5 phosphate | 0.05 |
| Isochem TPGS 1000 | Vitamin E TPGS | 0.5 |
| Acef 000595 | e.o. Thymus vulgaris L. | 0.05 |
| Acef 1191 | buffer q.s. to ph 7 | 0.3 |
| 1257 | | |

The eye drops were prepared according to the following protocol: add the water into a beaker then the riboflavin until completely dissolved.

Add vitamin E TPGS and wait for its complete solubilization. Add the thyme essential oil until completely incorporated. Separately prepare a buffer by adding 0.066 g of 0.025M dibasic potassium phosphate and 0.3 g of 0.025M monobasic phosphate in 100 g of water. Measure the pH and add the buffer drop by drop until reaching pH 7 (˜ 0, 3 g). The resulting solution was mixed and then placed in an appropriate dropper vial.

### Composition B

| Supplier + code | Qualitative composition | g in 100 g |
|---|---|---|
| Alsa Lab | Production Water | q.s. to 100 |
| Isochem TPGS 1000 | Vitamin E TPGS | 0.5 |
| Acef 000595 | e.o. Thymus vulgaris L. | 0.05 |
| Acef 1191 | buffer q.s. to ph 7 | 0.3 |
| 1257 | | |

The eye drops were prepared according to the following protocol: add the water to a beaker then the vitamin E and wait for its complete solubilization. Add the thyme essential oil until completely incorporated. Measure the pH and reach 7 with the buffer prepared as described for composition A (˜0.3 g). The solution was mixed and then placed in a special dropper vial.

### Composition C

| Supplier + code | Qualitative composition | g in 100 g |
|---|---|---|
| Alsa Lab | Production Water | q.s. to 100 |
| Acef 002925 | Riboflavin 5 phosphate | 0.05 |
| Acef 000595 | e.o. Thymus vulgaris L. | 0.05 |
| Acef 1191 | buffer q.s. to ph 7 | 0.3 |
| 1257 | | |

The eye drops were prepared according to the following protocol: add the water inside a beaker then the riboflavin and wait for its complete solubilization. Add the thyme essential oil until completely incorporated. Measure the pH and reach pH 7 with the buffer prepared as described for composition A. The solution was mixed and then placed in a appropriate dropper vial.

### Composition D

| Supplier + code | Qualitative Composition | g in 100 g |
|---|---|---|
| Alsa Lab | Production Water | Qs to 100 |
| Acef 000595 | e.o. Thymus vulgaris L. | 0.05 |
| Acef 1191 | buffer q.s. to ph 7 | 0.3 |
| 1257 | | |

The eye drops were prepared according to the following protocol: add the water to a beaker then add the thyme essential oil until completely incorporated. Measure the pH and reach pH 7 with the buffer prepared as described for composition A. The solution was mixed and then placed in an appropriate dropper vial.

### EXAMPLE 2: Medicated Wipes

Non-woven wipes were soaked in a sterile environment with solution A), B), C) or D) according to example 1 and sealed in aluminum sachets.

### Experimental Evidences

### Materials and Methods

### Materials

The solutions A, B, C and D of example 1 were tested, all based on thyme extract, in particular essential oil (e.o.) of thyme, in combination with riboflavin 5-phosphate and Vitamin E TPGS (composition A), in combination only with Vitamin E TPGS (composition B), in combination only with riboflavin 5-phosphate (composition C) and alone (composition D). Milli-Q deionized (DI) distilled water was used for all preparations.

### Cell Line and Culture Conditions

L929 fibroblast cell monolayers are highly representative of the target tissue in vivo. L929 cells were cultured in MEM (minimal essential medium) supplemented with fetal bovine serum (10%) and glutamine (4 mM) and incubated under standard culture conditions (37° C., 5% CO₂).

Ex Vivo Determination of the Corneal Penetration of Thyme or Thymol Extract into Franz Diffusion Cells

Corneal accumulation of the solution was assessed using a modified Franz-type diffusion cell (Ø 9 mm, receptor volume 5 mL, SES GmbH-Analysesysteme, Bechenheim, DE). Out of seven groups of 10 human corneas provided by Fast Linking (Fast Linking spin-off consortium, Naples, Italy), six were immersed for 20 minutes in a composition comprising thyme extract and VE-TPGS with increasing concentrations of VE-TPGS (compositions B1-B6) and labeled B1 to B6. A seventh group, labeled A, used as a control group, was immersed in the same solution as above without VE-TPGS (composition D). Corneas were treated as reported in Ostacolo, C.; Caruso, C.; Tronino, D.; Troisi, S.; Laneri, S.; Pacente, L.; Del Prete, A.; Sacchi, A. Enhancement of corneal permeation of riboflavin-5′-phosphate through vitamin E TPGS: A promising approach in corneal trans-epithelial cross linking treatment. Int. J. Pharm. 2013, 440, 148-153). Several iso-osmolar aqueous solutions buffered to pH 7.2 were used as the donor compartment. All contained 0.05% w/w of e.o. of thyme and different concentrations of VE-TPGS, as shown in Table 1. Solution A contained only e.o. of thyme and was used as a control. All solutions B1 to B6 were applied on epithelialized corneas in order to evaluate the accumulation of thymol as reported Ostacolo C., Caruso C., Tronino D., Troisi S., Laneri S., Pacente L., Del Prete A., Sacchi A. “Enhancement of corneal permeation of riboflavin-5′-phosphate through vitamin E TPGS: a promising approach in corneal trans-epithelial cross linking treatment. Int J Pharm. 2013; 440 (2): 148-153.)

### HPLC Analysis

The equipment and analytical settings were the same as used in [Ostacolo C., Caruso C., Tronino D., Troisi S., Laneri S., Pacente L., Del Prete A., Sacchi A. “Enhancement of corneal permeation of riboflavin-5′-phosphate through vitamin E TPGS: a promising approach in corneal trans-epithelial cross linking treatment. Int J Pharm. 2013; 440 (2): 148-153.-Viñ as P., Balsalobre N., López-Erroz C., Hernández-Córdoba M. Liquid chromatographic analysis of riboflavin vitamers in foods using fluorescence detection. J Agric Food Chem. 2004; 52 (7): 1789-1794.]. The results are expressed as mean±SEM. The data obtained were analyzed by Student's T test for statistical significance. A p value≤0.05 was considered significant in this study.

### In Vitro Toxicity by MTT Dosage

Composition B (i.e. comprising the combination VE TPGS and thyme extract) was tested for its toxicity on L929 cells by MTT assay [Stockert J. C., Horobin R. W., Colombo L. L., Blázquez-Castro A. “Tetrazolium salts and formazan products in Cell Biology: Viability assessment, fluorescence imaging, and labeling perspectives”, Acta Histochem. 2018; 120 (3): 159-167.]. Briefly, composition B comprising e.o. of thymol 0.05% and VE-TPGS 0.5% was incubated overnight in culture medium. An L929 cell suspension was prepared at a concentration of 4×104 cells/mL and seeded on 96-well culture plates (100 μL/each well). The cells were incubated in the medium containing the formulation or in subsequent dilutions (1:2) in culture medium for 24 hours. Sodium dodecyl sulfate (SDS) served as a positive control at concentrations ranging from 0.00313 to 0.4 mg/mL, while untreated cells in the culture medium were used as a negative control. After a 24 hour contact, the culture medium was carefully removed from each well and the cells were treated with 3-(4,5-dimethylthiazol-2-yl)-2,5-diphenyltetrazolium (or MTT) bromide (1 mg/mL) and incubated for 3 hours under standard culture conditions. After this period, the MTT solution was washed off and the formazan crystal was dissolved with 100 UL of isopropanol. The absorbance (optical density, OD) was determined spectrophotometrically at 540 nm wavelength.

### In Vivo Ocular Tolerance in Rabbits

A total of twelve (12) pathogen-free specific (SPF) white New Zealand female rabbits, approximately 11 weeks old, were ordered and supplied by Charles River Italia S.p.A., Calco (Lecco), Italy. The animals were assigned to two groups of 5 animals each, one control group receiving placebo only and the second receiving eye drops. Composition B, comprising respectively 0.5% and 0.05% of VE-TPGS and e.o. of thyme was instilled in the right eye, twice daily at an interval of 6 hours. A volume of 60 μL of the test eye drops (Composition B) or the control eye drops (Composition C) was instilled into the lower conjunctival sac of the right eye, ensuring that the globe or conjunctiva were not damaged. The eye was then kept closed for a few seconds to prevent loss of the formulation and favor distribution on the eye surfaces and conjunctival membranes. The left eyes served as a control. The animals were treated twice a day for 7 consecutive days. The animals were dosed up to the day before the necropsy. Both eyes of all animals were examined once pre-dose and twice daily after, before each administration. Any irritation observed was assigned a numerical value according to the table below based on the methods described by Draize et al. [Draize J.H., Woodard G., Calvery H.O. Methods for the study of irritation and toxicity of substances applied topically to the skin and mucous membranes. J. Pharmacol. Exp. Ther. 1944; 83, 377-390.]. The study complied with the ARVO Declaration for the use of animals in ophthalmic and visual research and in compliance with the guidelines of the European Economic Community for the care and welfare of animals (EEC Law No. 86/609). The study received approval from the Local Ethics Committee No. 21/2019.

### Microorganisms

The antimicrobial activity of composition B, i.e. comprising the combination VE-TPGS and e.o. of thyme according to the present invention, was evaluated against Staphylococcus aureus (ATCC 6538), S. epidermidis, Pseudomonas aeruginosa (ATCC 9027), Escherichia coli (ATCC 8739), Candida albicans (ATCC 10231) and Aspergillus niger (ATCC 1640).

### Assesment of MIC, MBC and MFC

To determine the minimum inhibitory concentrations (MICs) in vitro of composition B, i.e. comprising the combination e.o. of thyme and VE-TPGS, microbroth dilution tests were performed in line with CLSI (Clinical & Laboratory Standards Institute) guidelines [Wikler, M.A., Clinical and Laboratory Standard Institute. Performance Standards for Antimicrobial Susceptibility Testing. 18th Informational Supplement. Clinical and Laboratory Standards Institute, 2008. Wayne, PA]. In detail, from the nocturnal cultures of Mueller-Hinton Agar (Becton Dickinson and Company, Franklin Lakes, USA) the bacteria were first resuspended at a concentration of 1.5×10⁸ CFU/mL in sterile saline and subsequently diluted to a concentration of ˜ 1.5×10⁵ CFU/mL in cation-adjusted Mueller-Hinton broth (CAMHB, Thermo Scientific Waltham, USA) containing different dilutions of composition B, comprising the combination VE-TPGS and e.o. of thyme. The plates were incubated in air for 24 hours at 37° C.

Similarly, the fungistatic and fungicidal activity of composition B was tested against Candida albicans with the micro-broth dilution method in 96-well plates according to the guidelines reported in CLSI document M27-A3 [Clinical and Laboratory Standards Institute, CLSI, 2008. Reference Method for Broth Dilution Antifungal Susceptibility Testing of Yeasts; Approved Standard, third ed. Clinical and Laboratory Standards Institute, Wayne, PA CLSI Document M27-A3]. In detail, the colonies of the Sabouraud dextrose agar plate were cultured in prewarmed RPMI 1640 medium (with I-glutamine, 2% glucose, without bicarbonate and with phenol red as pH indicator) buffered to pH 7.0 with 0.165 M of morpholine propane sulfonic acid (MOPS). Several dilutions of composition B were added to each well containing 2×103 yeasts/ml. The plate was incubated at 37° C. for 24 or 48 hours.

The MIC was then determined visually as the lowest concentration showing no growth. MBC (minimum bactericidal concentration) and MFC (minimum fungicidal concentration) were determined by plating 20 μL of each well on agar plates incubated at 37° C. for 24 hours or 48 hours. MBC and MFC were identified as the lowest plated concentration to show no microbial growth. Each assay was performed in triplicate. The MBC/MIC ratios were calculated in order to determine the bactericidal/fungicidal (MBC/MIC≤4 or MFC/MIC≤4) or bacteriostatic/fungistatic (MBC/MIC>4 or MFC/MIC>4) effect of these compositions.

### Biofilm Inhibition

A protocol similar to antimicrobial experiments was used to determine the ability of composition B, i.e. comprising the combination of thyme extract and VE-TPGS referred to in the present invention, to inhibit the formation of bacterial biofilms. In detail, the bacteria after 24 hour incubation with different dilutions of the composition were washed twice with PBS to remove loosely adherent cells and then stained for 15 minutes with 200 μL of crystalline violet (CV, 0.3% w/v). The wells were then rinsed with water and dried. The amount of biofilm biomass was quantified by dissolving the 200 μL crystals of 33% acetic acid and then measuring the absorbance of the CV solution in a microplate spectrophotometer set at 600 nm. Untreated cells were used as controls. Each assay was performed in triplicate on separate days.

Similarly, in order to determine the ability of the formulation to inhibit C. albicans biofilm formation, yeast cells were inoculated in 7 mL of yeast nitrogen base (YNB) with 100 mM glucose (pH 7.0) and incubated overnight at 25° C. The culture is diluted to 10⁷ cells/mL and aliquoted in a 96-well flat-bottom plate (0.2 mL/well). C. albicans cells were allowed to adhere for 90 minutes at 37° C.; subsequently, the wells were washed with phosphate buffered saline (PBS, pH 7.2) to remove non-adherent cells. To allow adherent yeast cells to germinate and eventually form biofilms, a new YNB medium at pH 7.0 with different dilution (1:10) of the composition was added to each well and incubated at 37° C. After 48 hours, the biofilm mass was washed with PBS, dried, stained with CV for 15 minutes and quantified by retesting the wells with 200 ml of 33% acetic acid.

### Challenge Test

The microbial barrier properties of the composition were evaluated by an in vitro challenge test described by the European Pharmacopoeia to estimate potential contamination during its use. The formulation has been tested against the following bacteria and fungi: Staphylococcus aureus (ATCC 6538), Pseudomonas aeruginosa (ATCC 9027), Candida albicans (ATCC 10231) and Aspergillus niger (ATCC 16404). In detail, according to the standard methodology, the formulation was divided into 10 mL aliquots, which were inoculated with 106 and 105 colony forming units (CFU)/mL of bacteria and fungi respectively, and incubated between 20° C. and 25° C. The samples were collected in different time intervals: 2, 7, 14 and 28 days. 1 mL aliquots were serially diluted in typton-azolectin-tween broth and plated in duplicate on tryptic-soy agar (for bacteria) or Sabouraud dextrose agar (for fungi). The plates were incubated at 30° C. to 35° C. for ≥3 days for bacteria and between 20° C. and 25° C. for ≥5 days for fungi. Raw data counts have been converted to log 10 values.

### Results

### VE-TPGS Increases Corneal Accumulation of Thyme Extract or Thymol

To evaluate whether this new composition increases the bioavailability of thyme extract or thymol throughout the cornea, several compositions including the combination of thyme extract and VE-TPG of the present invention were applied on human corneas and analyzed by a system of diffusion cells of the modified Franz type. Specifically, seven groups of 10 human corneas were incubated for approximately 20 minutes with e.o. of thyme 0.05% w/w and an increasing percentage (w/w) of VE-TPGS (0 to 1), as shown in Table 1.

TABLE 1

| | % w/w | % w/w |
|---|---|---|
| Composition | e.o. of thyme | VE TPGS |
| A | 0.05 | |
| B1 | 0.05 | 0.010 |
| B2 | 0.05 | 0050 |
| B3 | 0.05 | 0.100 |
| B4 | 0.05 | 0.250 |
| B5 | 0.05 | 0.500 |
| B6 | 0.05 | 1.000 |

Then, the corneas were processed according to the procedure reported and the permeability of the e.o. of thyme was determined by analyzing the recipient's chamber over time and quantifying the molecule by HPLC [Ostacolo C., Caruso C., Tronino D., Troisi S., Laneri S., Pacente L., Del Prete A., Sacchi A. Enhancement of corneal permeation of riboflavin-5′-phosphate through vitamin E TPGS: a promising approach in corneal trans-epithelial cross linking treatment. Int J Pharm. 2013; 440 (2): 148-153.]. The accumulation of e.o. of thyme was expressed as nmol per mg of corneal tissue as reported in Table 2. Solution A was used as a control.

TABLE 2

| | thyme e.o. accumulation |
|---|---|
| Composition | (nmol/mg) |
| A | 0.032 ± 0.017 |
| B1 | 0.067 ± 0.035 |
| B2 | 0.184 ± 0.014 |
| B3 | 0.236 ± 0.021 |
| B4 | 0.346 ± 0.018 |
| B5 | 0.398 ± 0.034 |
| B6 | 0.453 ± 0.013 |

In all the analyzed conditions, except the one called B1, we measured a higher concentration of e.o. of thyme than the control (A). Interestingly, at increasing concentrations of VE-TPGS there was an increased accumulation of thyme e.o. up to condition B5 where VE-TPGS was used at 0.5% (w/w). No significant differences were found between conditions B5 (0.5% w/w VE-TPGS) and B6 (1% w/w VE-TPGS). The concentration of VE-TPGS should not exceed the critical concentration of micelles (CMC), since beyond a certain threshold the biocidal activity of the ophthalmic preservatives decreases significantly [Deluca P.P., Kostenbauder H. B. Interaction of preservatives with macromolecules IV. Binding of quaternary ammonium compounds by nonionic agents. J Am Pharm Assoc Am Pharm Assoc. 1960; 49:430-7.]. Furthermore, a higher concentration of non-ionic surfactants could carry a potential safety risk for disruption of the precorneal tear film. [Maurer N., Wong K. F., Hope M. J., Cullis P. R. Anomalous solubility behavior of the antibiotic ciprofloxacin encapsulated in liposomes: A 1H-NMR study. Biochim Biophys Acta. 1998; 1374 (1-2): 9-20.]. For these reasons, composition B5 was used for the following analysis.

### In Vitro Toxicity of the Composition VE-TPGS and Thyme Extract

Once a better bioavailability of e.o. of thyme, or thymol, through the cornea has been established, the composition comprising VE-TPGS and thyme extract was evaluated for its toxicity in vitro. A new collyrium is usually tested to be able to stimulate cell growth in vitro and to be compatible with their metabolism with non-cytotoxic effects, even at relatively high concentrations. The results would represent an index of the high skin compatibility of a medical device and would also suggest a potential role of the device itself in accelerating the flipping of the skin on which topical products are applied directly. [Rossi A. B., Bacquey A., Nocera T., Thouvenin M. D. Efficacy and Tolerability of a Medical Device Repairing Emollient Cream Associated with a Topical Corticosteroid in Adults with Atopic Dermatitis: An Open-label, Intra-individual Randomized Controlled Study. Dermatol Ther (Heidelb). 2019; 9 (2): 389.-Pinter A., Thouvenin M. D., Bacquey A., Rossi A. B., Nocera T. Tolerability and Efficacy of a Medical Device Repairing Emollient Cream in Children and Adults with Mild to Moderate Atopic Dermatitis. Dermatol Ther (Heidelb). 2019; 9 (2): 309-319.].

The cytotoxicity test was performed on L929 fibroblast cells which were treated with maceration liquid and subsequent 1:2 dilutions. The maceration liquid was obtained after incubating the formulation of VE-TPGS (0.5% w/w) and e.o. of thyme (0.05% w/w) in culture medium. After 24 hours of incubation, the cells were analyzed by MTT assay. As shown in Table 3, post-incubation cell viability with the extract (100% maceration liquid) was 88.4%, suggesting that this formulation neither cause cytotoxic effects nor stimulate cell proliferation.

TABLE 3

| Concentration | White | 100% | 50% | 25% | 12.50% | 6.25% | 3.13% | 1.56% | 0.78% |
|---|---|---|---|---|---|---|---|---|---|
| O.D. Media | 1.502 | 1.333 | 1.346 | 1.484 | 1.484 | 1.493 | 1.542 | 1.443 | 1.528 |
| Viability (%) | 100.00 | 88.42 | 89.33 | 99.02 | 98.78 | 99.37 | 102.74 | 95.94 | 101.78 |

### In Vivo Ocular Tolerance on Rabbits

The tolerance of the combination thyme extract and tocopherol was also tested in vivo by twice a day ocular instillation of 60 μL of composition B in the right eye of six rabbits for 7 days at the Research Toxicology Center (RTC STUDY NO. A3656). The left eye of each animal remained untreated and functioned as an intra-animal control. As a control group, another series of six rabbits were treated with 0.9% sodium chloride. Twice a day, the following parameters were observed and recorded in each animal: clinical signs, body weight, ocular observations (assessment of ocular irritation twice a day) and gross post-mortem macroscopic observations (with particular attention to the eye and annexes). None of the rabbits treated with the combination of thyme extract and tocopherol developed signs of systemic effects. Furthermore, the examination of both eyes before and after the dose of the combination did not reveal any effects related to the treatment or irritation. In fact, we assigned each animal a score of zero in a range between 0 and 4, where 0 corresponds to the normal physiology of the eye and 4 to the signs of increased irritation according to Draize et al. (Draize, J. H.; Woodard, G.; Calvery, H. O. Methods for the study of irritation and toxicity of substances applied topically to the skin and mucous membranes. J. Pharmacol. Exp. Ther. 1944, 83, 377-390). No treatment-related changes were observed in post mortem macroscopic observations. Together, the results strongly suggest that this new ophthalmic solution is well tolerated by the rabbit eye, particularly when administered twice a day at a dose of 60 μL on the surface of the eye.

### Antimicrobial Activity of the Collyrium Thyme Extract, or Thymol, and Tocopherol

In order to verify whether the antimicrobial activity of e.o. of thyme combined with VE-TPGS was preserved, MIC and MBC of the collyrium were evaluated against Gram-positive and negative strains of bacteria (Staphylococcus aureus, Staphylococcus epidermidis, Pseudomonas aeruginosa, Escherichia coli) and fungi (Candida albicans). As shown in Table 4, all bacteria except P. aeruginosa and C. albicans were significantly inhibited in their viability and among them, the strongest effect was against S. aureus with the lowest MIC (9 μL/mL) and MBC (35 μL/mL) values (Table 4). The MBC/MIC ratios showed that the formulation has a bactericidal and fungicidal effect on S. aureus, S. epidermidis and C. albicans respectively, while it is bacteriostatic on E. coli because the MBC/MIC ratio is higher than 4 (Table 4).

TABLE 4

| | | | Disk | | Biofilm |
|---|---|---|---|---|---|
| | MIC | MCB | diffusion | | Inhibition |
| Batterium | (μL/mL) | (μL/mL) | (10 μL) | | (μL/mL) |
| S. aureus | 9.0 | 35.0 | 10-15 | mm | 7.5 |
| S. epidermis | 27.5 | 75.0 | 0 | mm | 17.5 |
| E. coli | 30.0 | 40.0 | 0 | mm | 20.0 |
| P. aeruginosa | >100 μL/mL | >100 μL/mL | 0 | mm | >100 μL/mL |

| | MIC₀ | MFC |
|---|---|---|
| Fungus | (μL/mL) | (μL/mL) |
| C. albicans | 50.0 | 75.0 |

The ability of the formulation to prevent microbial growth was tested by the challenge test, in which the formulation was inoculated directly with S. aureus, P. aeruginosa, C. albicans and A. niger and their growth was monitored over time by counting their colonies on agar plates. Among the bacteria only S. aureus was completely inhibited all the time, while the fungi, C. albicans and A. niger, were reduced at two days by 3 times and 1 time respectively and at seven days by 5 times and 3 times respectively. At 14 days C. albicans never recovered, while A. niger maintained the same growth rate observed at seven days. These evidences suggest that the collyrium of the invention, i.e. comprising the combination of thyme extract, or thymol, and tocopherol, in particular VE-TPGS, meets the typical needs of antimicrobial eye drops.

In other words, these results demonstrate that despite the compositions comprising thyme extract, or thymol, are effective in the prevention and treatment of ocular diseases, in particular ocular infections, only the eye drops comprising the combination of the present invention, i.e. thyme extract or thymol together with tocopherol, in particular VE-TPGS, is suitable for enhancing the bioavailability of thymol in vivo. Furthermore, the experiments reported above have shown that the increase in bioavailability is directly proportional to the dose of tocopherol, in particular VE-TPGS.

In order to evaluate whether the combination according to any of the embodiments described here could represent a valid alternative to the antiseptic products known in the art to be useful in the treatment of ocular diseases, in particular ocular infections, further studies were conducted, in which different compositions based on thyme extract have been tested against Gram-positive (S. aureus and S. epidermidis) and Gram-negative (E. coli and P. aeruginosa) bacteria all closely related to different types of ocular infections such as blepharitis, conjunctivitis and keratitis. In addition, since many chronic eye infections are caused by protozoal parasites, the antiparasitic activity of various compositions based on thyme or thymol extract, in particular against A. castellanii, was also evaluated.

As will become clear from the following, thyme or thymol has shown surprising inhibitory effects especially against P. aeruginosa, often responsible for antibiotic-resistant ocular infections, and against Acanthamoeba castellanii, known to cause keratitis.

### Materials and Methods

The ophthalmic solutions tested and their respective formulations are shown in Table 5. Thymol was prepared at 500 mg/ml in ethanol (Sigma, T-0501, 99.5%).

TABLE 5
Ophthalmic solutions

0.75% Polyvinyl alcohol
0.75% Polyvinyl alcohol + 0.05% Thymol
0.75% Polyvinyl alcohol + 0.5% Thymol
0.5% Thymol

### Microorganisms

The antimicrobial activity of each ophthalmic composition was evaluated against: S. aureus (ATCC 6538), S. epidermidis (ATCC 03111), E. coli (ATCC 25922), P. aeruginosa (ATCC 9027) and Acanthamoeba castellanii (ATCC 50370). Microorganisms were obtained from LGC Standards S.r.L. (Milano, Italy).

### Determination of the Minimum Inhibitory Concentration and Minimum Bactericidal Concentration

To determine the minimum in vitro inhibitory concentration (MIC) of each solution, dilution assays of the micro-broth were performed according to the guidelines of the Clinical and Laboratory Standards Institute (CLSI). In detail, from “overnight” cultures in Mueller-Hinton Agar (Becton Dickinson and Company, Franklin Lakes, NJ, USA) the bacteria were resuspended to a concentration of ˜ 1.5×10⁸ CFU/ml in sterile saline and then further diluted at a concentration of ˜1.5×10⁵ CFU/ml in Mueller-Hinton Broth (MHB, Biolife SRL, Milan, Italy) containing different dilutions of each formulation listed in Table 5. The plates were incubated in air for 24 h at 37° C. The MICs were then determined visually by reading each bacterial culture in a spectrophotometer set at 600 nm. MIC₅₀ and MIC₁₀₀ were calculated as the lowest concentration (pl/ml) that caused 50 or 100% growth inhibition, respectively. The Minimum Bactericidal Concentration (MBC) was determined by plating 20 μl of each well on agar incubated at 37° C. for 24 h. MBC was identified as the lowest “platelet” concentration that did not show bacterial growth. Each assay was performed in triplicate.

TABLE 6
MIC₅₀ e MIC₁₀₀ of the ophthalmic solutions on S. aureus.

| Ophthalmic solutions | MIC₅₀ | MIC₁₀₀ |
|---|---|---|
| 0.75% PVA | n.d. | n.d. |
| 0.75% PVA + 0.05% thymol | 100 | n.d. |
| 0.75% PVA + 0.5% thymol | 50 | n.d. |
| 0.5% thymol | 50 | n.d. |

#MIC₅₀ e MIC₁₀₀ have been expressed as μl/ml.

In addition, 0.75% PVA, 0.75% PVA+0.05% thymol, and 0.75% PVA+0.5% thymol caused a reduction in the growth of S. epidermidis by up to 40% and a reduction in E. coli growth of 30% when used at 100 pl/ml. In particular, the two solutions comprising PVA and thymol reduced the growth rate of P. aeruginosa by 40% already at 50 μl/ml as well as PVA at 100 pl/ml. The latter result is particularly surprising considering that P. aeruginosa infections are difficult to treat even with antibiotics.

### Biofilm Inhibition

To determine the ability to inhibit bacterial biofilm formation, the bacteria were incubated with different dilutions of each of the formulations in Table 5. After 24 hours, the poorly adhered cells were removed by washing twice with Phosphate Buffered Saline (PBS) and biofilms were stained for 15 min with 200 μl of crystal violet (CV, 0.3% w/v). The biofilms were washed twice with water and then dissolved with 200 μl of 33% acetic acid. The amount of biomass was quantified by measuring the absorbance of the CV solution in a spectrophotometer set at 600 nm. Untreated cells were used as a control. Each assay was performed in triplicate.

### Agar Disk Diffusion Assay

In a modified agar disk diffusion assay, each microorganism from “overnight” cultures (with a concentration of approximately 2×10⁸ CFU/ml) was “plated” on Muller-Hinton Agar plates (MHB, Biolife SRL, Milan, Italy)) and then 5 μl of each formulation (Table 5) was directly deposited on the agar surface. After 24 hours of incubation at 37° C., a quantitative analysis of the antimicrobial effect was estimated as a growth inhibition zone. Also in this case, the thymol-based ophthalmic solutions listed in Table 5 have been shown to be effective in inhibiting bacterial growth.

### Amoebicidal Determination

Minimum trophozoite inhibition concentration (MTIC₅₀) is defined as 50% inhibition of Acanthamoeba castellanii trophozoite replication compared to controls. To determine the MTIC₅₀, two-fold serial dilutions of each formulation were made in Ringer's solution pH 7.4 and incubated with 100 μl of 2×104/ml axenic trophozoites in culture medium for approximately 48 hours at 25° C. At the end of the incubation, six photos per well were taken using the Digital Inverted Microscope AME-3206 (AMG/EVOS, Mill Creek, WA, USA) at 10× magnification and the degree of amoeba growth was determined in each of the conditions tested by counting the number of trophozoites. Furthermore, a qualitative analysis of the amoeba shape and the presence of cell lysis was evaluated by representing the experimental conditions at 40× magnification.

The solution comprising 0.5% thymol resulted in a 50% reduction in amoeba growth (MTIC₅₀).

### Statistical Analysis

Results were expressed as means±SD. The data were statistically analyzed using a t-test. A p value<0.05 was considered statistically significant.

1.-14. (canceled)

15. A method of treating ophthalmic diseases, comprising topically administering an effective amount of thymol or an extract of thymol in combination with about 0.001-0.400 μg per day of a tocopherol to the eyes of a patient in need thereof.

16. The method of claim 15, wherein the tocopherol is alpha-tocopherol or a polyoxyalkylene glycol ester of an alpha-tocopherol ester with a carboxylic acid.

17. The method of claim 16, wherein the alpha-tocopherol of said alpha-tocopherol ester with a carboxylic acid is selected from the group consisting of tocopherol succinate, tocopherol acetate, tocopherol linoleate and tocopherol phosphate.

18. The method of claim 16, wherein the polyoxyalkylene glycol moiety of said polyoxyalkylene glycol ester has a molecular weight between about 600 Da and about 6000 Da.

19. The method of claim 18, wherein the polyoxyalkylene glycol moiety of said polyoxyalkylene glycol ester has a molecular weight, preferably between about 600 Da and about 1500 Da.

20. The method of claim 16, wherein the polyoxyalkylene glycol ester of an alpha-tocopherol ester with a carboxylic acid is d-α-tocopherol polyethylene glycol 1000 succinate (VE-TPGS).

21. The method of claim 15, wherein extract of thymol is obtained from a plant belonging to the genus Thymus and is selected from the group consisting of Thymus vulgaris L. extract, Thymus zygis extract, Thymus striatus extract, Thymus spinulosus extract, Thymus richardii extract, Thymus catharinae extract, Thymus herba-barona extract, Thymus odoratissimus extract, Thymus oenipontanus extract, Thymus kosteletzkyanus extract, Thymus alpestris extract, Thymus pulegioides extract, Thymus longicaulis extract, Thymus praecox extract, Thymus thracicus extract and mixtures thereof.

22. The method of claim 21, wherein the extract of thymol is obtained from Thymus vulgaris L.

23. The method of claim 15, wherein the weight ratio of the thymol extract or thymol, and the tocopherol is between 0.001:4 and 0.01:3.

24. The method of claim 15, wherein the thymol extract or thymol is administered at a concentration between 0.001 and 1% w/w.

25. The method of claim 24, wherein the thymol extract or thymol is administered at a concentration between 0.01 and 0.1% w/w.

26. The method of claim 25, wherein the thymol extract or thymol is administered at a concentration of 0.05% w/w.

27. The method of claim 15, wherein the tocopherol or and ester of tocopherol is administered at a concentration between 0.01 and 3% w/w.

28. The method of claim 27, wherein the tocopherol or and ester of tocopherol is administered at a concentration between 0.1 and 2% w/w.

29. The method of claim 28, wherein the tocopherol or and ester of tocopherol is administered at a concentration 0.5% w/w.

30. The method of claim 15, further comprising administering an active ingredient selected from the group consisting of: beta blockers; alpha-agonists; carbonic anhydrase inhibitors; dopaminergic agonists and antagonists; cholinergic drugs; prostaglandins; ACE inhibitors; steroids; calcium channel blockers; antihypertensives; non-steroidal anti-inflammatory drugs; steroidal anti-inflammatory drugs; antibiotics; antifungals; antivirals; anti-cataract agents; antioxidants; antihistamines; antimetabolites; immunosuppressants; growth factors, vitamins, preferably riboflavin-5-phosphate, and mixtures thereof.

31. The method of claim 15, wherein the combination is administered as an isotonic solution, buffered at pH 7, optionally containing polyvinyl alcohol.

32. The method of claim 15 wherein the combination is administered as one of a collyrium, solution, suspension, eye drops, spray, ophthalmic bath, gel, salve, ointment, cream, medicated wipe, occlusive dressing or ophthalmic inserts.

33. The method of claim 15, wherein the ocular diseases are ocular infections.

34. The method of claim 33, wherein the ocular infection is caused by Pseudomonas aeruginosa or Acanthamoeba castellanii.

35. The method of claim 33, wherein the ocular infection is selected from the group consisting of chalazion, sty, blepharitis, keratitis, actinic keratitis, conjunctivitis, uveitis, and endophthalmitis.

36. The method of claim 15, wherein the ocular disease is selected from the group consisting of glaucoma, cataract, keratoconus, dry eye, conjunctival allergy, damage from UV exposure, maculopathy, meibomitis, trabeculitis, corneal ulcers, and optic neuritis.

37. The method of claim 15, wherein the thymol or thymol extract is administered separately from the tocopherol or simultaneous therewith.

38. A method of treating ophthalmic diseases, comprising orally administering an effective amount of thymol or an extract of thymol in combination with about 0.001-0.400 μg per day of a tocopherol to a patient in need thereof, wherein the combination is administered sequentially or simultaneously and wherein the thymol or extract of thymol and the tocopherol are administered in a form selected from the group consisting of capsules, soft capsules, tablets, pills, lozenges, gelatines, powders, granules, emulsions, solutions, suspensions, syrups and elixirs.
